(19) United States
(12) Patent Application Publication
(10) Pub. No.: US 2003/0228283 A1
(43) Pub. Date: Dec. 11, 2003

(54) Preventing secondary lymphedema with VEGF-D DNA

(75) Inventors: Lucie Margarete Heinzerling, Zurich (CH); Megan Elizabeth Baldwin, Brunswick (AU); Steven Alan Stacker, North Fitzroy (AU); Maro Gregory Achen, North Melbourne (AU)

Correspondence Address: CROWELL & MORING LLP, INTELLECTUAL PROPERTY GROUP, P.O. BOX 14300, WASHINGTON, DC, 20044-4300, US

(73) Assignee: Ludwig Institute for Cancer Research, New York, NY

(21) Appl. No.: 10/424,992

(22) Filed: Apr. 29, 2003

Related U.S. Application Data
(60) Provisional application No. 60/377,253, filed on May 3, 2002.

(51) Int. Cl. A61K 48/00

(52) U.S. Cl. 424/93.2; 514/44

(57) ABSTRACT

Methods of preventing secondary lymphedema with DNA encoding VEGF-D and/or VEGF-D protein, and biologically active fragments and analogs thereof, as well as pharmaceutical compositions for treating secondary lymphedema, are presented.

### CROSS REFERENCE TO RELATED APPLICATION

This application claims priority of U.S. Provisional Application No. 60/377,253 filed May 3, 2002.

### FIELD OF THE INVENTION

The present invention relates generally to the fields of molecular biology and medicine; more particularly to the areas of treatment, prevention, or inhibition of secondary lymphatic disorders, and more particularly to the treatment of secondary lymphedema by administration of VEGF-D DNA and/or protein.

### BACKGROUND OF THE INVENTION

The lymphatic system is a complex structure organized in parallel fashion to the circulatory system. In contrast to the circulatory system, which utilizes the heart to pump blood throughout the body, the lymphatic system pumps lymph fluid using the contractility of the lymphatic vessels. This lymphatic vasculature contributes to the regulation of interstitial fluid pressure in tissues by transporting excess fluid back into the circulation. Edema represents an imbalance between lymph formation and its absorption into the lymphatic vessels. A clinical condition of major importance is lymphedema that can arise due to impaired lymphatic drainage.

Lymphedema can be a disfiguring condition due to accumulation of lymph, or fatty fluid, within the tissues resulting in limb and tissue engorgement. The final result can be severely incapacitating due to local infections, sclerosis of the skin, discomfort and deformity.

Such lymphedema can be either primary or secondary. Primary lymphedema, also known as Milroy's disease, is hereditary. Secondary lymphedema, by contrast, is not hereditary, and may be caused by inflammatory or neoplastic obstruction of lymphatic vessels, and includes accumulation of ascites fluid due to peritoneal carcinomatosis or edema of the arm or other limbs following surgery or radiotherapy for breast cancer and other tumor types. Secondary lymphedema may also be idiopathic in origin. The present invention is directed particularly to the treatment of any type of secondary lymphedema.

At present, lymphedema is treated by manual lymphatic drainage and by compressive garments. The discovery of specific genes involved in the regulation of lymphatic vessels and in the pathology of lymphedema has made the design of more targeted treatments for this disease possible.

Specifically, two growth factors, named vascular endothelial growth factors C and D (VEGF-C and VEGF-D, respectively) due to amino acid sequence similarity to earlier-discovered vascular endothelial growth factor, have been shown to bind to and to activate tyrosine phosphorylation of the receptor Flt-4 (Achen, M. G. et al., 1998, Proc. Natl. Acad. Sci, USA 95: 548-553.). Transgenic overexpression of VEGF-C or VEGF-D has been shown to be able to induce the postnatal growth of new lymphatic vessels in the skin (Jeltsch et al., 1997, Science 276: 1423-1425; Veikkola et al., 2001, EMBO J. 20: 1223-1231).

VEGF-C was isolated from conditioned media of the PC-3 prostate adenocarcinoma cell line (CRL1435) by screening for ability of the medium to produce tyrosine phosphorylation of the endothelial cell-specific receptor tyrosine kinase VEGFR-3 (Flt-4), using cells transfected to express VEGFR-3. Its isolation and characteristics are described in detail in Joukov et al., EMBO J., 1996 15: 290-298.

VEGF-D was isolated from a human breast cDNA library, commercially available from Clontech, by screening with an expressed sequence tag obtained from a human cDNA library designated “Soares Breast 3NbHBst” as a hybridization probe (Achen et al., 1998, Proc. Natl. Acad. Sci. USA 95: 548-553). Its isolation and characteristics are described in detail in International Patent Application No. PCT/US97/14696 (WO 98/07832).

The VEGF-D gene is broadly expressed in the adult human, but is certainly not ubiquitously expressed. VEGF-D is strongly expressed in heart, lung and skeletal muscle. Intermediate levels of VEGF-D are expressed in spleen, ovary, small intestine and colon, and a lower expression occurs in kidney, pancreas, thymus, prostate and testis. No VEGF-D mRNA was detected in RNA from brain, placenta, liver or peripheral blood leukocytes.

Subcutaneous adenoviral gene transfer of VEGF-C in mice has been shown to induce lymphangiogenesis within two weeks of treatment (Enholm et al., 2001, Circ. Res. 88: 623-629). A mouse model (Chy), which mimics human hereditary lymphedema, allows the study of potential gene therapies (Karkkainen et al., 2001, Proc. Natl. Acad. Sci. USA 98, 12677-12682). When VEGF-C was overexpressed in the skin of Chy mice, growth of functional cutaneous lymphatic vessels was induced, suggesting that VEGF-C or VEGF-D gene therapy may be applicable to primary human lymphedema.

While such therapy may have been hypothesized for the treatment of non-hereditary, regional forms of lymphedema (resulting from surgery or lymphatic vessel destruction after cancer therapy), VEGF-D had not previously been tested in the context of secondary lymphedema.

### SUMMARY OF THE INVENTION

The instant invention establishes for the first time the usefulness of DNA encoding VEGF-D for inhibition, treatment, or prevention of secondary lymphedema, particularly DNA in a plasmid.

The instant invention also provides methods of inhibition, treatment, or prevention of secondary lymphedema with VEGF-D protein, or a biologically active fragment or analog thereof.

The invention provides a method of treating secondary lymphedema by stimulation of angiogenesis, lymphangiogenesis, neovascularization, connective tissue development and/or wound healing in a mammal in need of such treatment, comprising administering to the mammal an effective dose of DNA encoding VEGF-D, or a fragment or an analog thereof which has the biological activity of VEGF-D. One exemplary fragment is the VEGF homology domain (VHD, also called VEGF-DΔNΔC), which encodes residues 93 to 201 (inclusive) of human VEGF-D.

The invention also provides a method of treating secondary lymphedema by stimulation of angiogenesis, lymphangiogenesis, neovascularization, connective tissue development and/or wound healing in a mammal in need of such treatment, comprising administering to the mammal an effective dose of VEGF-D protein, or a fragment or an analog thereof which has the biological activity of VEGF-D. One exemplary fragment is the VEGF homology domain (VHD, also called VEGF-DΔNΔC), which contains residues 93 to 201 (inclusive) of human VEGF-D.

One aspect of the present invention provides a method of stimulation of lymphangiogenesis in a mammal in need of such treatment.

Optionally the DNA encoding VEGF-D, or VEGF-D protein, or fragment or analog thereof, may be administered together with, or in conjunction with, one or more of VEGF, VEGF-B, VEGF-C, PlGF, PDGF-A, PDGF-B, PDGF-C, PDGF-D, FGF, and heparin. One example is a VEGF-D/VEGF-C heterodimer. Another example is a VEGF-D/VEGF-C heterodimer wherein the dimer comprises the VHD domains. Any VEGF molecules may be of mammalian or viral origin, and in one embodiment are of human or mouse origin.

The VEGF-D protein, or fragment or analog thereof, may be in the form of a monomer or a dimer, wherein the dimer may be a homodimer of VEGF-D, or may be a heterodimer with at least one of VEGF, VEGF-B, VEGF-C, PlGF, PDGF-A, PDGF-B, PDGF-C, PDGF-D, FGF, and heparin.

A “patient” includes any mammal, and in one embodiment of the present invention is a human.

The dose(s) and route(s) of administration will depend upon the form of the VEGF-D (protein or DNA, excipient used, etc.), on the nature of the patient and condition to be treated, and will be at the discretion of the attending physician or veterinarian. Suitable routes include oral, subcutaneous, intramuscular, intraperitoneal, intradermal, or intravenous injection. Parenteral or topical application, implants, etc., may be employed in combination with a suitable pharmaceutical carrier to effectuate administration to a patient in need of such treatment.

For intramuscular preparations, a sterile aqueous formulation, preferably of a suitably soluble form of the DNA encoding VEGF-D can be dissolved and administered in a pharmaceutical diluent, such as pyrogen-free water (distilled), physiological saline, or 5% glucose solution. A suitable insoluble form of the compound may be prepared and administered as a suspension in an aqueous base or a pharmaceutically acceptable oil base, e.g., an ester of a long chain fatty acid such as ethyl oleate.

The resulting compositions comprise a therapeutically effective amount of DNA encoding VEGF-D or a biologically active fragment thereof, and a pharmaceutically acceptable excipient. Other compositions comprise a therapeutically effective amount of VEGF-D protein or a biologically active fragment thereof.

These compositions may be administered by any method known in the art that is effective for delivery of such protein or DNA. Acceptable delivery routes include the use of viruses and viral vectors, particularly viruses and viral vectors which have been genetically modified so as to deliver genes to target cells in a patient without adverse infectious reactions. Of particular interest as delivery means are DNA viruses. Adenoviruses, herpesviruses, parvoviruses, and avipox viruses are examples of suitable delivery vectors, though other viruses may be used.

Administration may also be via liposomes, or via various polymeric carriers such as polyols and optionally derivatized or modified RNA, DNA, or protein (see, for example, U.S. Pat. No. 6,312,727 to synthetic polymer based carrier materials). Multilayer compositions, such as cochleates or other lipid bilayer derived structures are also useful for administration.

“DNA encoding VEGF-D or a biologically active fragment or analog thereof” means isolated DNA sequences encoding the isolated proteinaceous growth factor, vascular endothelial growth factor D, which has the ability to stimulate and/or enhance proliferation or differentiation of endothelial cells. An example of a biologically active fragment is VEGF-DΔNΔC, and tagged versions such as VEGF-DΔNΔC-FLAG, as described and synthesized in Stacker, S. A., et al., Biosynthesis of vascular endothelial growth factor-D involves proteolytic processing which generates non-covalent homodimers, J. Biol. Chem. (1999) 274: 32127-32136.

These sequences include conservative substitutions that do not change the biological activity of native VEGF-D. Also included are DNA sequences which encode possible variant forms of the VEGF-D polypeptide which may result from alternative splicing, as are known to occur with VEGF and VEGF-B, and also naturally-occurring allelic variants of the nucleic acid sequence encoding VEGF-D. Allelic variants are well known in the art, and represent alternative forms of a nucleic acid sequence which comprise substitution, deletion, or addition of one or more nucleotides, but which do not result in any substantial functional alteration of the encoded polypeptide.

Such variant functional forms of VEGF-D can be prepared by targeting non-essential regions of the VEGF-D polypeptide for modification and modifying the originating DNA accordingly (by processes well known to those of skill in the molecular biological arts). These non-essential regions are expected to fall outside the strongly-conserved regions.

In particular, the growth factors of the PDGF/VEGF family, including VEGF, are dimeric, and VEGF, VEGF-B, VEGF-C, VEGF-D, PlGF, PDGF-A and PDGF-B show complete conservation of eight cysteine residues in the PDGF/VEGF-like domains (Olofsson et al., 1996, Proc. Natl. Acad. Sci. USA, 93: 2576-2581; Joukov et al., 1996, EMBO J., 15: 290-298). These cysteines are thought to be involved in intra- and inter-molecular disulfide bonding.

In addition there are further strongly, but not completely, conserved cysteine residues in the C-terminal domains. Loops 1, 2, and 3 of each VEGF homology domain (VHD) subunit, which are formed by intra-molecular disulfide bonding, are involved in binding to the receptors for the PDGF/VEGF family of growth factors (Andersson et al., 1995, Growth Factors, 12: 159-164).

Persons skilled in the art thus are well aware that these cysteine residues should be preserved in any proposed functional variant form, and that the active sites present in loops 1, 2, and 3 also should be preserved. However, other regions of the molecule can be expected to be of lesser importance for biological function, and therefore offer suitable targets for modification. Modified polypeptides can readily be tested for their ability to show the biological activity of VEGF-D by routine activity assay procedures such as the endothelial cell proliferation assay.

Also within the scope of the invention are analogs of VEGF-D that have altered receptor binding specificity.

An “excipient” according to the present invention includes solid or liquid carrier or adjuvants, examples of which include, but are not limited to, saline, buffered saline, Ringer's solution, mineral oil, talc, corn starch, gelatin, lactose, sucrose, microcrystalline cellulose, kaolin, mannitol, dicalcium phosphate, sodium chloride, alginic acid, dextrose, water, glycerol, ethanol, thickeners, stabilizers, suspending agents, and combinations thereof. These excipients also may include any necessary buffering, chelating, or salt agents, including TRIS (tris(hydroxymethyl)aminomethane) and EDTA (ethylene diamine tetraacetic acid). Any suitable DNA delivery vehicle known in the art may be used, including various physiological solutions and liposomes.

Compositions according to the present invention may be in the form of solutions, suspensions, tablets, capsules, creams, salves, elixirs, syrups, wafers, ointments, or other conventional forms, so long as the form does not react unfavorably with the VEGF-D active ingredient. The formulation is adjusted to suit the mode of administration. Compositions of the present invention may optionally further comprise one or more of PDGF-A, PDGF-B, PDGF-C, PDGF-D, VEGF, VEGF-B, VEGF-C, PlGF, and heparin.

Compositions comprising DNA encoding VEGF-D will contain from about 0.1% to 90% by weight of the active compound(s), and most generally from about 10% to 30%. Generally, a typical active dosage of VEGF-D protein, or DNA encoding VEGF-D protein, will be within the rage of about 1 ng to about 10 mg.

As used herein, the term “conservative substitution,” when used in the context of DNA, includes substitutions which may be made because of the degeneracy of the genetic code; i.e., where more than one DNA codon encodes the same amino acid. This term also encompasses substitutions made for codon optimization, i.e., where certain codon replacements are made so as to optimize protein expression in a particular species.

As used herein, the term “conservative substitution,” when used in the context of a polypeptide, denotes the replacement of an amino acid residue by another, biologically similar residue. Examples of conservative substitutions include the substitution of one hydrophobic residue such as isoleucine, valine, leucine, alanine, cysteine, glycine, phenylalanine, proline, tryptophan, tyrosine, norleucine or methionine for another, or the substitution of one polar residue for another, such as the substitution of arginine for lysine, glutamic acid for aspartic acid, or glutamine for asparagine, and the like. Neutral hydrophilic amino acids which can be substituted for one another include asparagine, glutamine, serine and threonine. The term “conservative substitution” also includes the use of a substituted amino acid in place of an unsubstituted parent amino acid.

As such, it should be understood that in the context of the present invention, a conservative substitution is recognized in the art as a substitution of one amino acid for another amino acid that has similar properties. Exemplary conservative substitutions are set out in the following Table A from WO 97/09433.

TABLE A
Conservative Substitutions I

| SIDE CHAIN CHARACTERISTIC | AMINO ACID |
|---|---|
| Aliphatic Non-polar | G A P I L V |
| Polar - uncharged | C S T M N Q |
| Polar - charged | D E K R |
| Aromatic | H F W Y |
| Other | N Q D E |

Alternatively, conservative amino acids can be grouped as described in Lehninger, Biochemistry, 2nd Ed.; Worth Publishers, Inc. NY:N.Y. (1975), pp.71-77, as set out in the following Table B.

TABLE B
UZ,8/25 Conservative Amino Acid Substitutions II

| SIDE CHAIN CHARACTERISTIC | AMINO ACID |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

Exemplary conservative substitutions are set out in the following Table C.

TABLE C
Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met (M) | Leu, Phe, Ile |
| Phe (F) | Leu, Val, Ile, Ala |
| Pro (P) | Gly |
| Ser (S) | Thr |
| Thr (T) | Ser |
| Trp (W) | Tyr, Phe |
| Tyr (Y) | Trp, Phe, Thr, Ser |
| Val (V) | Ile, Leu, Met, Phe, Ala |

### BRIEF DESCRIPTION OF THE DRAWINGS

FIG. 1 is a graph showing the development of lymphedema over time in mouse tails after surgery and treatment with plasmid encoding VEGF-DΔNΔC (VEGF-D) or parental expression vector (Apex-3). The experiment was performed as set forth in Example 1.

FIG. 2 is a photograph showing the tails of three mice treated with Apex-3 (empty vector, upper section) and with vector encoding VEGF-DΔNΔC (lower section), 13 days after surgery and plasmid injection. The sites of surgery are indicated by arrows. The experiment was performed as set forth in Example 1.

### DETAILED DESCRIPTION OF PREFERRED EMBODIMENTS

### EXAMPLE 1

Materials and Methods

Induction of lymphedema: Mice used were strain C57/Black 6 and were from six to ten weeks of age. Induction of secondary lymphedema in the mouse tail was achieved by ligation of the lymphatics with a circumferential incision 1 cm along the tail from the tail-base, broadly as described previously (Slavin, S. A. et al., 1999, Ann. Surg. 229: 421-427). The incision was cauterized and the gap in the tissue was filled with two-component fibrin sealant (TISSEEL®, Baxter Hyland Immuno, Vienna, Austria). Lymphedema was quantified by measurement of the diameter of the tail at various distances distal to the incision using digital calipers. Mice reproducibly developed lymphedema over ten days.

Generation of plasmids: A region of the human VEGF-D cDNA was inserted into the mammalian expression vector Apex-3 (Evans et al., Mol. Immunol., 1995 32 1183-1195). This vector is maintained episomally when transfected into 293-EBNA human embryonal kidney cells. For expression of mature VEGF-D (spanning from amino acid residues 93-201 of human VEGF-D), the region of pEFBOSVEGF-DΔNΔC containing the sequences encoding the IL-3 signal sequence, the FLAG® octapeptide and the mature VEGF-D were inserted into the XbaI site of Apex-3 (see Example 9 in International Patent Application PCT/US97/14696 (WO98/07832)).

The resulting plasmid was designated pVDΔpexΔNAC (Stacker, S. A. et al., 1999, J. Biol Chem. 274: 32127-32136, and see Example 1 in International Patent Application PCT/US98/27373). The entire disclosure of the International Patent Application PCT/US98/27373 is incorporated herein by reference

Treatment with plasmid DNA. Plasmid DNA (pVDApexΔNΔC) encoding the mature form of human VEGF-D, tagged at the N-terminus with the FLAG octapeptide (the encoded protein designated VEGF-DΔNΔC) was prepared using EndoFree Plasmid Mega kits (available from QiagenGmbH, Germany), though any plasmid preparation can generally be used. The plasmid DNA was injected at the side of incision immediately after cauterization. Negative control plasmid was the parental expression vector Apex-3 lacking any sequence encoding VEGF-D. Both plasmids were delivered by four intradermal injections (50 μg/injection), two on each side of the incision. DNA injections were carried out immediately prior to application of fibrin sealant that had been mixed with approximately 50 μg of plasmid DNA before use.

Results

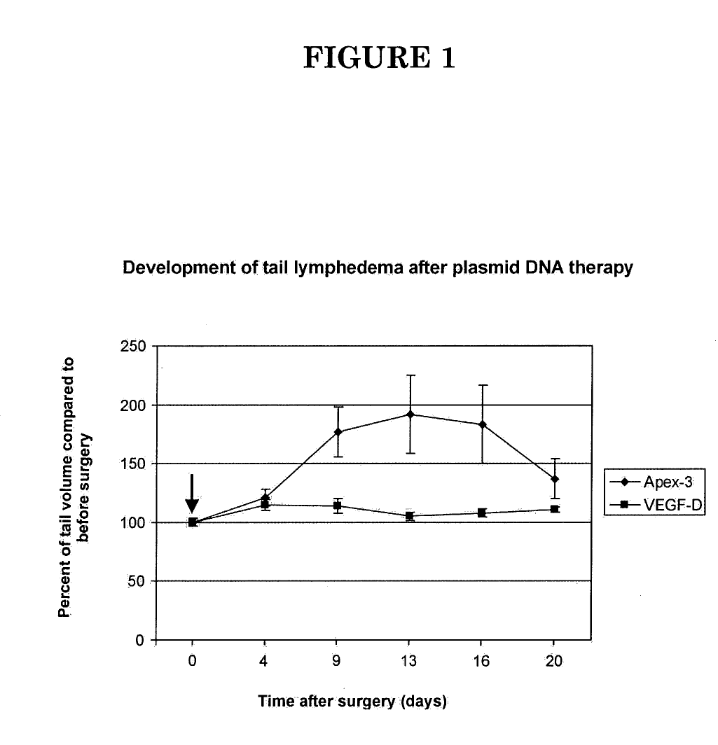

Mice were injected with plasmid DNA encoding the mature form of human VEGF-D (VEGF-DΔNΔC), or with parental expression vector, Apex-3, as negative control. Lymphedema developed reproducibly in mice injected with the negative control, being most severe 13 days after surgery, with tail volume almost doubling during that period (FIG. 1). In FIG. 1, the arrow indicates the time at which surgery and plasmid injection were carried out. Data points represent the mean and error bars the standard error. The 100% value was established by measuring tails immediately before surgery, and both study groups consisted of five mice.

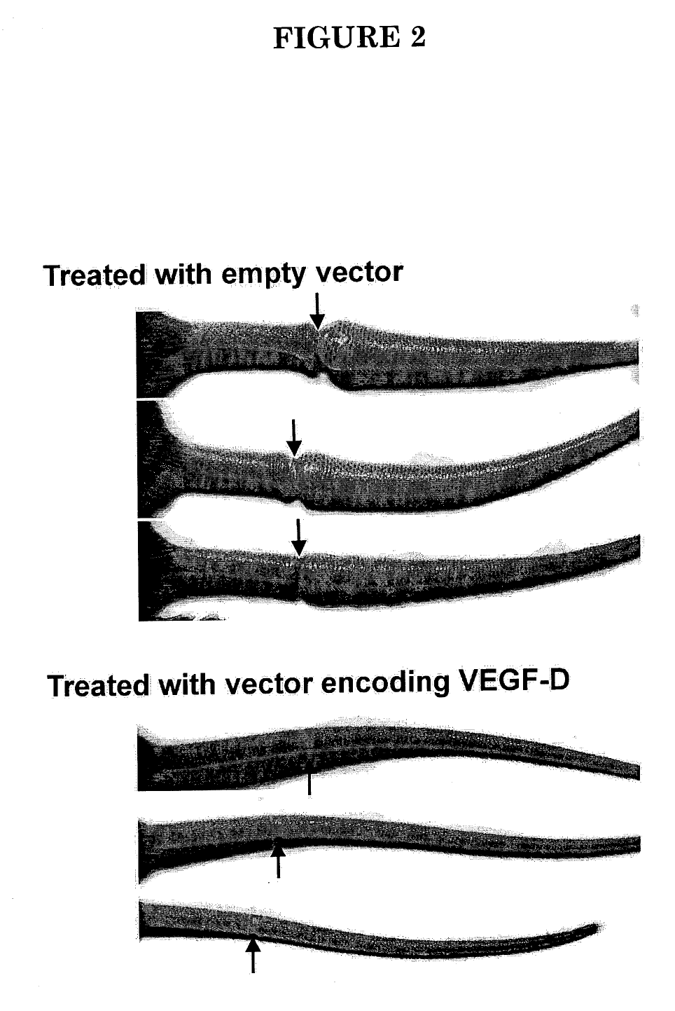

The result shown in FIG. 1, following injection with Apex-3, was comparable to that obtained when no plasmid DNA was injected (data not shown). In contrast, animals injected with plasmid encoding VEGF-DΔNΔC developed only very moderate lymphedema by day 4 which subsequently resolved, presumably due to formation of dermal lymphatics that establish connection with deeper draining lymphatic vessels. A photograph comparing the tails of mice from both treatment groups 13 days after surgery and plasmid injection is shown in FIG. 2. The arrows indicate the incision site. The absence of lymphedema from the mice treated with VEGF-DΔNΔC plasmid is apparent.

### EXAMPLE 2

Patients suffering from lymphatic spread of the primary tumor, e.g. patients with melanoma or breast cancer, often undergo lymphadenectomy to remove tumor from lymphatics and lymph nodes. Radiotherapy is used to further eradicate tumor cells from the lymph nodes in these patients. These interventions frequently induce lymphedema. These patients are treated with the compositions and methods of the invention in order to inhibit or treat the lymphedema.

Initially, plasmid DNA encoding human VEGF-DΔNΔC is injected axillary or inguinally in patients with established lymphedema in a dose-escalating scheme (for example, 100 μg-200 μg-500 μg-1000 μg-2000 μg) in order to determine the maximum tolerated dose (MTD). After evaluation for safety, plasmid DNA encoding human VEGF-DΔNΔC (at dosages at or below the MTD) is injected at the time of surgery in order to prevent formation of lymphedema.

Similarly, injection of plasmid DNA encoding human VEGF-DΔNΔC is also used to treat idiopathic lymphedema.

The foregoing description and examples have been set forth merely to illustrate the invention and are not intended to be limiting. Since modifications of the disclosed embodiments incorporating the spirit and substance of the invention may occur to persons skilled in the art, the invention should be construed broadly to include all variations falling within the scope of the appended claims and equivalents thereof.

We claim:

1. A method of treating secondary lymphedema, comprising:
administering to a patient with secondary lymphedema a therapeutically effective amount of DNA encoding vascular endothelial growth factor-D (VEGF-D).

2. A method of claim 1, wherein said patient is human

3. A method of claim 2, wherein said DNA is circular.

4. A method of claim 3, wherein said DNA is a plasmid.

5. A method of treating secondary lymphedema, comprising:
administering to a patient with secondary lymphedema a therapeutically effective amount of DNA encoding VEGF-D,
wherein said secondary lymphedema is caused by at least one of inflammatory obstruction of lymphatic vessels, neoplastic obstruction of lymphatic vessels, accumulation of ascites fluid due to peritoneal carcinomatosis, and edema of at least one limb.

6. A method of claim 5, wherein said edema follows at least one of surgery and radiotherapy for a neoplastic disease.

7. A method of claim 6, wherein said neoplastic disease is breast cancer.

8. A method of claim 6, wherein said neoplastic disease is at least one tumor type.

9. A method of inhibiting secondary lymphedema, comprising:
administering to a patient at risk for secondary lymphedema a therapeutically inhibitory amount of DNA encoding VEGF-D.

10. A method for expressing VEGF-D in a target cell, comprising
selecting a plasmid capable of expressing DNA encoding VEGF-D, wherein said plasmid has at least one insertion site for insertion of said DNA encoding VEGF-D operably linked to a promoter capable of expression in said target cell;
inserting said DNA encoding VEGF-D into said insertion site, and introducing said plasmid into said target cell wherein said DNA encoding VEGF-D is expressed at detectable levels.

11. A method of treating secondary lymphedema, comprising:
administering to a patient with secondary lymphedema a therapeutically effective amount of at least one plasmid encoding VEGF-DΔNΔC.

12. A method of treating secondary lymphedema, comprising:
administering to a patient with secondary lymphedema a therapeutically effective amount of vascular endothelial growth factor-D (VEGF-D) protein.

13. A method of claim 12, wherein said patient is human

14. A method of treating secondary lymphedema, comprising:
administering to a patient with secondary lymphedema a therapeutically effective amount of VEGF-D protein, wherein said secondary lymphedema is caused by at least one of inflammatory obstruction of lymphatic vessels, neoplastic obstruction of lymphatic vessels, accumulation of ascites fluid due to peritoneal carcinomatosis, and edema of at least one limb.

15. A method of claim 14, wherein said edema follows surgery or radiotherapy for a neoplastic disease.

16. A method of claim 15 wherein said neoplastic disease is breast cancer.

17. A method of claim 15, wherein said neoplastic disease is at least one tumor type.

18. A method of inhibiting secondary lymphedema, comprising:
administering to a patient at risk for secondary lymphedema a therapeutically preventative amount of VEGF-D protein.

19. A method of claim 1, wherein said DNA encoding VEGF-D is administered to said patient by at least one DNA virus.

20. A method of claim 1, wherein said DNA encoding VEGF-D is administered to said patient by at least one of an adenovirus, a herpesvirus, a parvovirus, and an avipox virus.

21. A method of claim 1, wherein said DNA encoding VEGF-D is administered to said patient by at least one liposome.

22. A method of claim 1, wherein said lymphedema is idiopathic.

23. A method of claim 12, wherein said lymphedema is idiopathic.
